(19) United States
(12) Patent Application Publication
(10) Pub. No.: US 2009/0155770 A1
(43) Pub. Date: Jun. 18, 2009

(54) IMPLANTABLE DEVICES FOR FIBER OPTIC BASED DETECTION OF NOSOCOMIAL INFECTION

(75) Inventors: Tameka Brown, Lilburn, GA (US); Akosua Atta-Mensah, Bethesda, MD (US); Daniel Baird, Woodstock, GA (US); Richard Hantke, Chicago, IL (US); Tod Hoover Shultz, Killingworth, CT (US); Erica M. Phillips, Woodstock, GA (US); Shawn R. Feaster, Duluth, GA (US); Mike Rainone, Palestine, TX (US); Thomas Edward Plowman, Cary, NC (US); Talbot Presley, Palestine, TX (US)

Correspondence Address: DORITY & MANNING, P.A., POST OFFICE BOX 1449, GREENVILLE, SC, 29602-1449, US

(73) Assignee: Kimberly-Clark Worldwide, Inc., Neenah, WI (US)

(21) Appl. No.: 11/954,881

(22) Filed: Dec. 12, 2007

(51) Int. Cl. C12Q 1/70

(52) U.S. Cl. 435/5

(57) ABSTRACT

Disclosed are methods and devices for continuous in vivo monitoring of a potential infection site. Disclosed devices may be utilized to alert patients and/or health care providers to the presence of a pathogen at an early stage of a hospital acquired infection, thereby providing for earlier intervention and improved recovery rates from bacterial infection. Disclosed methods utilize implantable devices for location at an in vivo site. The implantable device is held in conjunction with an optical fiber that detects and transmits an optically detectable signal generated in the presence of a pathogen. Upon generation of the emission, the optically detectable emission signal may be transmitted to a portable detection/analysis device. Analysis of the characteristics of the emission signal produced may be used to determine the presence or concentration of pathogens at the site of inquiry, following which real time information may be transmitted to medical personnel, for instance via a wireless transmission system.

### BACKGROUND

Nosocomial or hospital acquired infections (HAI) have been estimated by the World Health Organization (WHO) to kill between 1.5 and 3 million people every year worldwide. Though commonly referred to as hospital acquired infections, nosocomial infections result from treatment in any healthcare service unit, and are generally defined as infections that are secondary to the patient's original condition. In the United States, HAIs are estimated to occur in 5 percent of all acute care hospitalizations, resulting in more than $4.5 billion in excess health care costs. According to a survey of U.S. hospitals by the Centers for Disease Control and Prevention (CDC), HAIs accounted for about 1.7 million infections and about 99,000 associated deaths in 2002. The CDC reported that “[t]he number of HAIs exceeded the number of cases of any currently notifiable disease, and deaths associated with HAIs in hospitals exceeded the number attributable to several of the top ten leading causes of death in U.S. vital statistics” (Centers for Disease Control and Prevention, “Estimates of Healthcare Associated Diseases,” May 30, 2007).

HAIs, including surgical site infections (SSIs), catheter related blood stream infections (CRBSIs), urinary tract infections (UTIs), ventilator associated pneumonia (VAP), and others, may be caused by bacteria, viruses, fungi, or parasites. For instance, bacterial organisms, such as Escherichia coli, Staphylococcus aureus, and Pseudomonas aeruginosa are common causes as are yeasts such as Candida albicans and Candida glabrata, fungi such as those of the genus Aspergillus and those of the genus Saccharomyces, and viruses such as parainfluenza and norovirus.

Ongoing efforts are being made to prevent HAI through, for instance, improved hand washing and gloving materials and techniques, but such efforts have met with limited success. In an effort to better understand and curb HAIs, government regulations have increased pressure on hospitals and care-givers to monitor and report these types of infections. However, these measures are further complicated due to the prevalence of outpatient services, a result of which being that many HAIs do not become evident until after the patient has returned home. As such, infection may proceed undiagnosed for some time, complicating treatment and recovery.

A need currently exists for improved methods for diagnosing HAI, including SSI. Moreover, methods that could monitor a patient, for instance a patient's surgical site, in an outpatient setting, would be of great benefit.

### SUMMARY

In accordance with one embodiment, disclosed is a method for detecting the presence or amount of a pathogen that is a source of a hospital acquired infection. For example, a method may include locating a portion of an implantable device in an in vivo environment. A method may also include transmitting an optically detectable signal that is directly or indirectly emitted from the pathogen through a fiber optic cable to a detector. For instance, bacterial pathogens may autofluoresce in response to an excitation signal and directly produce the optically detectable signal. The presence or amount of the pathogen may then be determined.

According to another embodiment, a portable device for detecting the presence or amount of a pathogen that is a source of a hospital acquired infection is disclosed. A device may include, for instance, an implantable device and a portable enclosure containing a power source, an optical detector, a signal processor, and a signaling device for emitting a signal upon detection of the pathogen in an environment. The device may also include a connecting device, for instance for attaching the enclosure to the clothing or body of a wearer. In addition, the device may include the fiber optic cable that is affixed to the implantable device and that may be in optical communication with the detector and may extend from the enclosure, so as to be inserted into the environment of interest. Accordingly, disclosed devices may provide for improved monitoring of potential infection sites with little or no additional burden on health care workers.

Other features and aspects of the present disclosure are discussed in greater detail below.

### BRIEF DESCRIPTION OF THE DRAWINGS

A full and enabling disclosure of the subject matter, including the best mode thereof, directed to one of ordinary skill in the art, is set forth more particularly in the remainder of the specification, which makes reference to the appended figures in which:

FIG. 1 illustrates one embodiment of a composite sensing device as disclosed herein;

FIG. 2 illustrates an end portion of one embodiment of a sensing device as disclosed herein;

FIG. 3 illustrates a cross-sectional view of one embodiment of a composite sensing device as disclosed herein;

FIGS. 4A-4E illustrate illustrative examples of optical fiber designs that are encompassed in the present disclosure;

FIGS. 5A-5C are schematic representations of a fiber optic cable as may be incorporated in a device as disclosed herein;

FIG. 6 illustrates another embodiment of a composite sensing device as disclosed herein;

FIG. 7 illustrates another embodiment of a composite sensing device as disclosed herein;

FIG. 8 schematically illustrates one embodiment of a portable signal detection device as may be utilized with a composite sensing device as disclosed herein;

FIG. 9 illustrates another embodiment of a composite sensing device as disclosed herein;

FIG. 10 illustrates another embodiment of a composite sensing device as disclosed herein;

FIG. 11 illustrates another embodiment of a composite sensing device as disclosed herein;

FIG. 12 illustrates another embodiment of a composite sensing device as disclosed herein;

Repeat use of reference characters in the present specification and drawings is intended to represent same or analogous features or elements.

### DETAILED DESCRIPTION OF REPRESENTATIVE EMBODIMENTS

Reference now will be made in detail to various embodiments of the disclosed subject matter, one or more examples of which are set forth below. Each example is provided by way of explanation, not limitation. In fact, it will be apparent to those skilled in the art that various modifications and variations may be made in the present disclosure without departing from the scope or spirit of the subject matter. For instance, features illustrated or described as part of one embodiment may be used on another embodiment to yield a still further embodiment. Thus, it is intended that the present disclosure covers such modifications and variations as come within the scope of the appended claims and their equivalents.

The present disclosure is generally directed to methods for detection of HAI. In one particular embodiment, disclosed methods may be utilized for continuous monitoring of a potential infection site and may be utilized to alert patients and/or health care providers to the presence of pathogens at an early stage of infection, thereby providing for earlier intervention and improved recovery rates from infection.

Any source of HAI may be detected according to disclosed methods. In one particular embodiment, common bacterial sources such as Escherichia coli, Staphylococcus aureus, and Pseudomonas aeruginosa may be detected. However, it should be understood that disclosed methods are not limited to either these bacteria or bacterial pathogens in general. Other common sources of HAI that may be detected according to disclosed methods include, without limitation, other bacterial sources such as coagulase-negative staphylococci, Enterococcus spp., Enterobacter spp., Klebsiella pneumoniae, Proteus mirablis, Streptococcus spp., and so forth, as well as yeast, fungal, viral, and parasitic sources, as previously mentioned.

Presently disclosed methods and devices utilize a fiber optic-based sensor to examine a potential infection site or fluid obtained there from for the presence of HAI pathogens. More specifically, disclosed sensors include a fiber optic cable for transmitting an optically detectable signal from a site of inquiry that is provided in conjunction with an implantable medical device. The optically detectable signal may carry information regarding the presence or amount of a pathogen at the site that is a cause of an HAI.

An optically detectable signal that may signify the presence of a pathogen may be generated according to any methodology. For instance, in one particular embodiment, an optically detectable signal may be directly generated by a pathogen, e.g., a pathogenic bacterium upon autofluorescence of the pathogen. According to this embodiment, when in the presence of an excitation signal, a pathogen may autofluoresce with a unique spectral signature. An excitation signal may be provided via the same fiber optic cable that transmits the optically detectable signal from the site or may be provided from a different source, as desired. Analysis of the characteristics of the emission signal produced in response to the excitation signal may be used to determine the presence or concentration of pathogens at the site of inquiry and provide a route for early detection of a nosocomial infection.

It should be understood, however, that the method of generating the optically detectable signal is not critical to the disclosed subject matter, and disclosed devices may be utilized to detect any optically detectable signal produced either directly or indirectly due to the presence of the pathogen in the local environment. For instance, in another embodiment, disclosed devices may be utilized in conjunction with a fluorescent dye including a material that may specifically bind a targeted pathogen. Such fluorescent dyes are known in the art and may be utilized in conjunction with a sensor device as disclosed herein. For instance, U.S. Pat. No. 5,545,535 to Roth, et al., U.S. Pat. No. 5,573,909 to Singer, et al., U.S. Pat. No. 6,051,395 to Rocco, and U.S. Patent Application Publication No. 2007/0086949 to Prasad, et al. disclose fluorescent materials that may exhibit specific binding to a targeted material, for instance a targeted surface receptor of a bacterium. In the presence of the targeted bacteria, the fluorescent dyes may bind the bacteria and emit an optically detectable signal. Thus, the pathogen may indirectly produce the detectable signal.

Irrespective of the manner of generation of the optically detectable signal, upon generation of the signal, a sensor device as disclosed herein, including a fiber optic cable in conjunction with an implantable medical device, may detect and transmit the signal. Beneficially, disclosed sensing devices may incorporate any implantable medical device in conjunction with a fiber optic cable. For instance, a sensing device may include a fiber optic cable in conjunction with an implantable catheter. As utilized herein, the term ‘implantable catheter’ generally refers to an elongated structure that may be either flexible or rigid for insertion into a body cavity, duct, or vessel to allow the passage of fluids either into or out of the body or to distend a passageway. In addition, an implantable catheter may define one or more lumens therein.

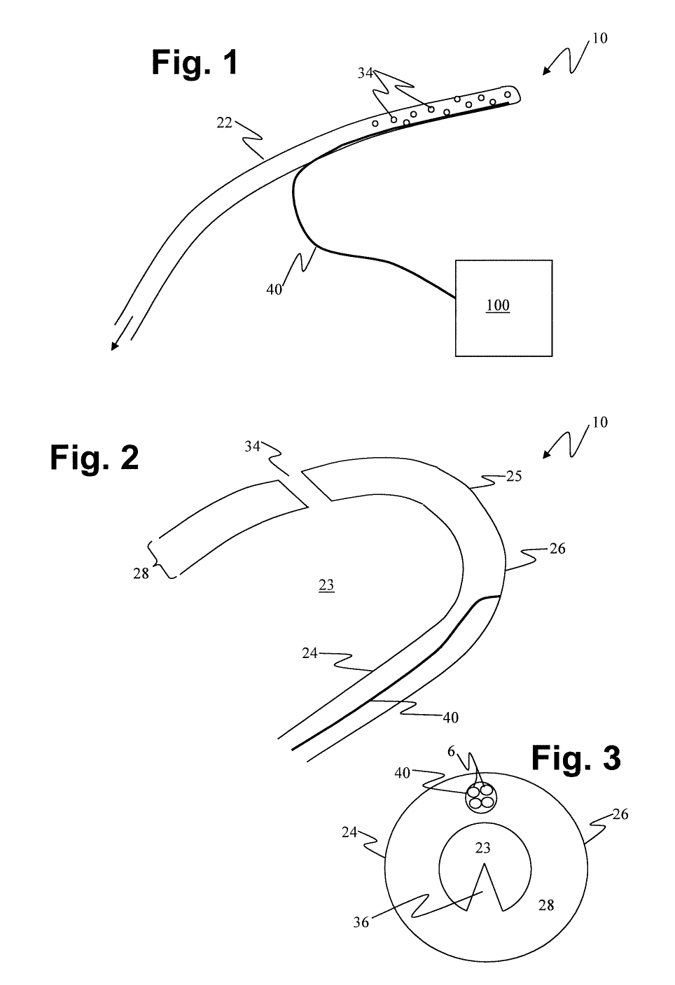

Referring to FIG. 1, one embodiment of a sensing device 10 is illustrated. Device 10 includes a catheter 22 and fiber optic cable 40. Catheter 22 includes a series of apertures 34, for instance as may be utilized to carry fluid away from a wound, surgical site, or other potential in vivo infection site, as is illustrated by the directional arrow of FIG. 1. Catheter 22 may be made of any material suitable for implantation. Beneficially, catheter 22 may be formed of biocompatible materials that may remain at a site of interest for a relatively long period of time, for instance to monitor the site for infection throughout the healing process and until high potential for infection has past. By way of example, catheter 22 may be of a biocompatible silicone, latex rubber, polyvinyl chloride (PVC), polyurethanes, Teflon®, any medical grade elastomer, and so forth. Catheter 22 may be of any color, and may be entirely or partially transparent.

An end portion of device 10 is illustrated in more detail in FIG. 2. As may be seen, catheter 22 includes inner surface 24 and an outer surface 26 that define a catheter wall 28 extending from the inner surface 24 to the outer surface 26. In addition, aperture 34 extends from the inner surface 24 to the outer surface 26, such that fluid may pass through aperture 34 and into lumen 23. In addition, fiber optic cable 40 is held in conjunction with wall 28 along a length of catheter 22. For instance, fiber optic cable 40 may be embedded within wall 28 either during for following formation of catheter 22. Alternatively, fiber optic cable 40 may be secured to inner surface 24 or outer surface 26 of catheter 22. For instance, fiber optic cable 40 may be secured to outer surface 26 by use of a medical grade adhesive. For example, biocompatible adhesives based upon proteins such as gelatins may be utilized, as may those formed from polysaccharides. Fiber optic cable 40 may alternatively be affixed to catheter 22 through use of solvent bonding, thermal bonding, ultrasonic bonding, and so forth.

At least one end of fiber optic cable 40 may terminate at outer surface 26 of catheter 22. Accordingly, light may pass into and/or out of fiber optic cable 40 at the terminus of fiber optic cable 40. The other end of fiber optic cable 40 is at a monitor 100, which is described in more detail below.

FIG. 3 illustrates a cross-sectional view of device 10. Though illustrated in FIG. 3 as having a generally circular cross-section, catheter 22 may have any cross-sectional shape, including rectangular, round, oval, and so forth. As may be seen, in this embodiment, inner surface 24 of catheter wall 28 defines an internal structure 36 that extends into the lumen 23. Internal structure 36 may extend partially or completely across the entire width of lumen 23 and may extend down the axial length of catheter 22 for any length. While not a requirement of disclosed devices, internal structure 36 may be beneficial in certain embodiments, for instance to provide additional strength to catheter 22. For example, internal structure 36 may prevent catheter wall 28 from collapsing should catheter 22 be subject to vacuum forces, as from a suction device, and/or strong lateral compression forces due to, e.g., motion of the wearer or others. In this particular embodiment, fiber optic cable 40 is a multi-fiber cable including a plurality of optical fibers 6 and is embedded within wall 28 of catheter 22.

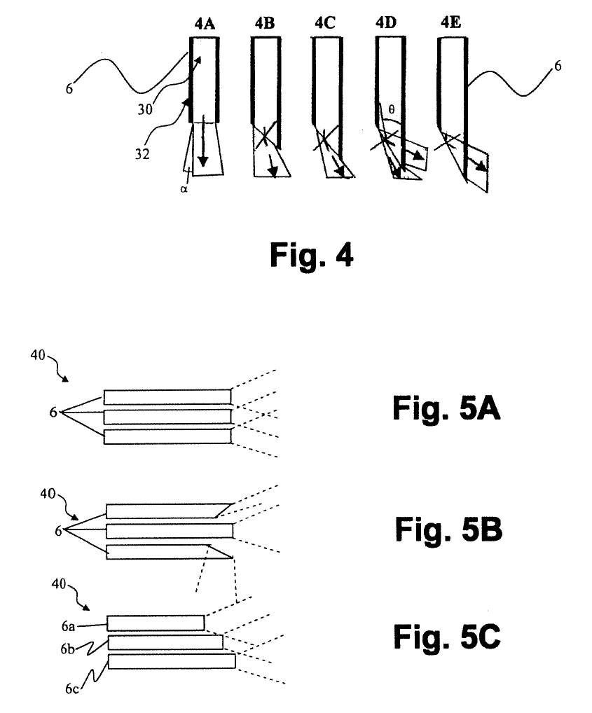

Optical fibers and cables may have a variety of physical characteristics, depending upon the specific requirements of a sensing system and site of inquiry. FIG. 4 schematically illustrates several embodiments of optical fibers as may be utilized in a sensing device. In general, an optical fiber 6 may include a core 30, through which light may travel, and an external cladding layer 32. The difference in the index of refraction between the core material and the clad material defines the critical angle θ at which total internal reflection takes place at the core/clad interface. Thus, light that impinges upon the interface at an angle greater than the critical angle is completely reflected, allowing the light to propagate down the fiber.

Optical fibers for use as disclosed herein may generally include multi-mode fibers having a core diameter greater than about 10 micrometers (μm). The preferred core diameter in any particular embodiment may depend upon the characteristics of a signal that is expected to travel through the fiber, among other system parameters. For instance, in those embodiments in which a laser excitation source is used to deliver an excitation signal through an optical fiber, a core diameter may be between about 50 μm and about 100 μm, or about 80 μm in one embodiment. In other embodiments, for instance in those embodiments in which an excitation light source produces less coherent radiation, such as a multi-wavelength light emitting diode (LED), for example, it may be preferable to utilize an optical fiber for carrying the excitation signal that defines a somewhat larger core diameter, for instance between about 90 μm and about 400 μm.

The core/clad boundary of a fiber may be abrupt, as in a step-index fiber, or may be gradual, as in a graded-index fiber. A graded-index fiber may be preferred in some embodiments, as graded index fibers may reduce dispersion of multiple modes traveling through the fiber. This is not a requirement however, and step-index fibers may alternatively be utilized, particularly in those embodiments in which an optical fiber is of a length such that dispersion will not be of great concern.

An optical fiber may be formed of sterilizable, biocompatible materials that may be safely placed and held at a potential infection site, and in one particular embodiment, at a surgical site. For example, an optical fiber formed of any suitable type of glass may be used, including, without limitation, silica glass, fluorozirconate glass, fluoroaluminate glass, any chalcogenide glass, or so forth may form the core and/or the clad.

Polymer optical fibers (POF) are also encompassed by the present disclosure. For instance, an optical fiber formed of suitable acrylate core/clad combinations, e.g., polymethyl methacrylates, may be utilized. It may be preferred in some embodiments to utilize a multi-core POF so as to lower losses common to POF due to bending of the fiber. For instance, this may be preferred in those embodiments in which an optical fiber may be located at an in vivo site of inquiry in a non-linear conformation.

The end of a fiber may be shaped as desired. For instance, and as illustrated in FIGS. 4A-4E, polishing or otherwise forming a specific angle at the end face of a fiber may maintain the acceptance angle α and collection efficiency of the fiber, while rotating the field of view of the fiber, as depicted by the arrows on FIGS. 4A-4E. Depending upon the angle at the fiber end, light may enter the fiber from angles up to about 90° of the fiber axis (e.g., as shown at FIG. 4E) (see, e.g., Utzinger, et al., Journal of Biomedical Optics, 8(1):121-147, 2003).

An optical fiber may be formed so as to detect an emission signal at locations along the length of the fiber, in addition to at the terminal end of the fiber. For instance, at locations along the length of the fiber the clad may be etched, generally with a predetermined angle, such that excitation light may exit the fiber and/or detectable signals emitted from a pathogen may enter the optical fiber at these locations. For example, the clad of a fiber may be bent or otherwise notched at a predetermined angle to form a ‘window’ in the fiber. Thus, a single optical fiber may detect signals from transformed bacterial over a larger area.

A fiber optic-based sensor for use as described herein may include a fiber optic cable comprised of a single optical fiber or a plurality of individual fibers, depending upon the specific design of the sensor. For instance, a plurality of optical fibers may be joined to form a single fiber cable of a size to be combined with an implantable device and located at an in vivo site of interest. For instance, a multi-fiber fiber optic cable may have a diameter of less than about 1.5 mm. Moreover, when considering utilization of a multi-fiber fiber optic cable, it may be beneficial to utilize a portion of the optical fibers of the cable to deliver an excitation signal to an area, while other optical fibers of the cable may be utilized to carry emission signals from the area back to a detection device.

When utilizing a plurality of optical fibers in a fiber bundle or cable, individual fibers may be formed and arranged in relation to one another so as to provide a wider field of detection. For instance, FIGS. 5A-5C illustrate several different embodiments of a fiber cable 40 comprising multiple optical fibers 6 in the bundle. As shown at FIG. 5A, through location of a plurality of fiber ends at a single cross-sectional area, improved light collection may be attained, as the total field area covered by the combined fibers will be larger than that for a single fiber. In the embodiment illustrated in FIG. 5B, the geometry of the end face of different fibers contained in the cable 40 may be different from one another, so as to allow light collection from a variety of different directions.

In the embodiment illustrated in FIG. 5C, fiber ends are staggered over a length, so as to increase the axial length of the light collection area and increase the area of inquiry in an axial direction. For instance optical fiber 6a may transmit one or more excitation signals to an area proximal to the terminus of fiber 40. The excitation signal may be specifically predetermined so as to excite autofluorescence in one or more HAI-causing pathogens. Accordingly, second and third optical fibers 6b and 6c may collect light from the site, which may include autofluorescent signals of pathogens present in the area that have been excited by the excitation signal, and return those signals to a detector for analysis.

Of course, combinations of such designs, as well as other fiber design for improving the collection of a signal area, including methods as discussed above as well as methods as are generally known to those in the art, may be utilized as well.

A sensor may be located at a site according to any suitable method. For instance, in the embodiment illustrated in FIG. 1, the end of sensing device 10 including the illustrated terminal portion of catheter 22 and the terminal portion of fiber optic cable 40 affixed thereto may be located at an in vivo site of interest during a medical procedure. In one particular embodiment, the site of interest may be a surgical site and the terminal portion of the sensor may be located within all or a portion of the surgical site prior to closing of the surgery. In another embodiment, the terminal portion of the sensor may be located within a wound site, for instance during cleaning, dressing changing, and so forth, at the wound site.

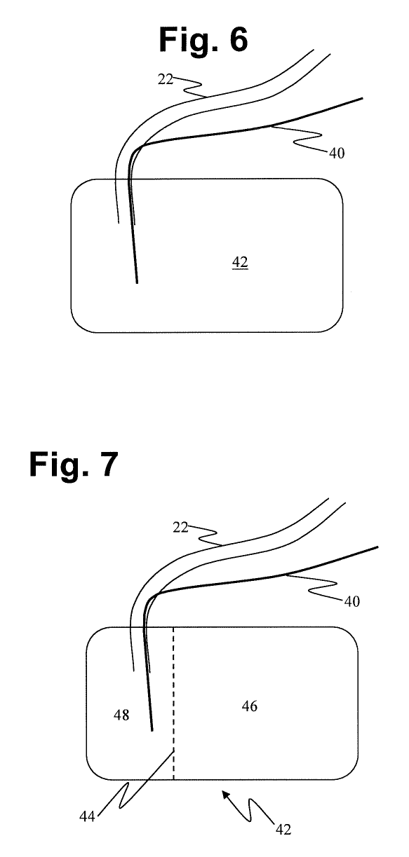

FIG. 6 schematically illustrates another embodiment of a sensor system as disclosed herein. According to this particular embodiment, the external end of catheter 22 may include a collection reservoir 42. For instance, catheter 22 may function as a wound or surgical site drain as described above and may drain fluid from an in vivo site into reservoir 42. Catheter 22 may also be associated with a suction device (not shown) as is known in the art for providing improved drainage to the system. In addition, fiber optic cable 40 may be associated with catheter 22 at the external end of catheter 22. For instance, fiber optic cable 40 may affixed to and extend from the end of catheter 22 such that fiber optic cable passes into reservoir 42 in conjunction with catheter 22 and extends beyond the end of catheter 22 and into reservoir 42.

Reservoir 42 may be of any suitable size and material as is known in the art. For instance, reservoir 42 may be formed of the same materials as catheter 22. In one embodiment, reservoir 42 may be formed of an opaque material, so as to limit excessive background light during the detection regime.

Fluid may pass through catheter 22 and into reservoir 42 and be held in reservoir 42 for a period of time. During that time, an optically detectable signal from a pathogen contained in the fluid held in reservoir 42 may be detected through utilization of a sensing device as disclosed herein. For example, one or more excitation signals may be transmitted from fiber optic cable 40 to illuminate fluid held in reservoir 42. The excitation signal(s) may be predetermined so as to induce targeted pathogens in the fluid to autofluoresce. Reflection and any emission signals generated due to the presence of pathogens in the fluid may than be transmitted via fiber optic cable 40 from reservoir 42 to a detection device and analyzed for unique spectral signatures indicative of HAI-causing pathogens. A system such as that illustrated in FIG. 6 may effectively examine fluid from a large in vivo area for the presence of HAI-causing pathogens.

Another embodiment of a detection system as disclosed herein is illustrated in FIG. 7. According to this embodiment, pathogens contained in a fluid from an in vivo site, e.g., a wound, a surgical site, and so forth, may be further filtered and concentrated, so as to improve detection of the pathogens. More specifically, reservoir 42 may include a porous barrier 44 therewithin. For instance, Barrier 44 may be a semi-permeable barrier defining a porosity that may allow smaller components of a fluid, e.g., smaller proteins and so forth, to be filtered out and pass through barrier 44 and into portion 46 of reservoir 42, while preventing passage of larger materials across barrier 44. Thus, larger materials may be held and concentrated in portion 48 of reservoir 42. For instance, barrier 44 may prevent a bacterial pathogen from passage, and thus larger materials may be captured, concentrated and filtered in portion 48 of reservoir 42. For instance, the concentration of bacterial pathogens within portion 48 may exceed about 10⁵ colony forming units per milliliter (CFU/ml). The concentration of bacteria in portion 48 may be greater in other embodiments, for instance greater than about 10⁶ CFU/ml, or greater than about 10⁷ CFU/ml, in another embodiment. According the spectral signatures of pathogens contained in the fluid held in portion 48 may be more easily differentiated from background noise.

Barrier 44 may be, for instance, a semi-permeable porous membrane having a porosity to allow materials less than about 0.2 μm across the membrane, with a preferred pore size generally depending upon the size of pathogens that are targeted for detection. By way of example, semi-permeable membrane 44 may be derived from a water insoluble, water wettable cellulose derivative, such as cellophane, cellulose acetate, cellulose propionate, carboxyethyl cellulose, and so forth; insolubilized gelatin; partially hydrolized polyvinyl acetate; or polyionic film forming compositions such as polysulfonated anionic polymers or ionically linked polycationic polymers, such as marketed by Amicon Company. Barrier 44 may be attached to a wall of reservoir 42 or optionally may be attached to another component of a sensing system. Fiber optic cable 40 may terminate at a location with portion 48 of reservoir 42 so as to examine the fluid for the presence of HAI-causing pathogens.

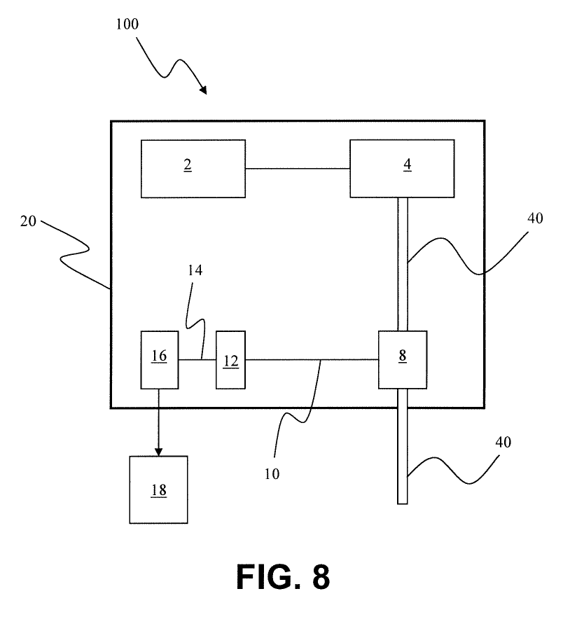

Referring to FIG. 8, a monitor 100 may be incorporated with a sensing device as disclosed herein. As may be seen in FIG. 8, monitor 100 may include several components that may be housed within an enclosure 20.

In one preferred embodiment, enclosure 20 may be portable. For example, enclosure 20 may be a molded plastic enclosure of a size so as to be easily carried by or attached to a wearer. For instance, enclosure 20 may include clips, loops, or so forth so as to be attachable to a patient's clothing or body. In one embodiment, enclosure 20 may include an adhesive surface, and may be adhered directly to a patient's skin. In general, enclosure 20 may be relatively small, for instance less than about 10 cm by about 8 cm by about 5 cm, so as to be inconspicuously carried by a patient and so as to avoid impedance of a patient's motion. Enclosure 20 may completely enclose the components contained therein, or may partially enclose the components contained therein. For example, enclosure 20 may include an access port (not shown) that may provide access to the interior of enclosure 20. In one embodiment, an access port may be covered with a removable cover, as is known in the art.

A first component as may be held within enclosure 20 is power supply 2 that may be configured in one embodiment to supply power to an excitation source 4 as well as other of the operational components as will be later described. In an exemplary configuration, power supply 2 may correspond to a battery, however those of ordinary skill in the art will appreciate that other power supplies may be used including those that may be coupled to an external alternating current (AC) supply so that the enclosed power supply may include those components necessary to convert such external supply to a suitable source for the remaining components requiring a power source.

As previously noted, power supply 2 may be configured to supply power to excitation source 4. In the illustrated exemplary configuration, excitation source 4 may correspond to a light emitting diode (LED), however, again, such source may vary and may include, but is not limited to, laser diodes and incandescent light sources. Excitation source 4 may correspond to a white light source, a non-white multi-wavelength source, or a single wavelength source, as desired or required. In a preferred exemplary configuration, an LED may be selected due to the low power consumption of such sources. The wavelength of the excitation energy supplied by excitation source 4 may be of any suitable wavelength, from infrared (IR) to ultraviolet (UV). In general, the preferred excitation energy wavelength may depend upon any specific pathogens for which the device is designed to detect. For instance, in those embodiments in which a specific bacteria or genera is being detected, the excitation wavelength may be specific for that target. In other embodiments, however, for instance when a plurality of different pathogens are being detected, and the different pathogens respond to different excitation wavelengths, an excitation source may provide multiple wavelengths, either through combination of signals from a plurality of single wavelength sources or through a single, incoherent source, as desired.

Excitation energy source 4 is optically coupled to a fiber optic cable 40 as illustrated. Fiber optic cable 40 is configured to extend externally from enclosure 20 to the field of inquiry, e.g., within a surgical site or other wound, and so forth. It should be appreciated that although the monitor 100 of FIG. 8 is illustrated as including only a single fiber optic cable 40, such is not a specific limitation of the present disclosure as such devices may, in fact, include multiple fiber optic cables. For instance, different fiber optic cables may be utilized for delivering an excitation signal and receiving an emission signal from different areas of a site. Those of ordinary skill in the art will appreciate that a single excitation energy source may be optically coupled to a plurality of optical fibers and/or a plurality of fiber optic cables through utilization of suitable beam splitters, mirrors, and so forth.

Moreover, as discussed previously, plural excitation energy sources may be used. In such a configuration, each excitation source may be optically coupled to one or more optical fibers and or fiber optic cables such that multiple excitation wavelengths may be delivered to the field of enquiry.

Housed within enclosure 20 is an optical detector 8 coupled to fiber optic cable 40. Optical detector 8 may correspond to a photodiode, a photoresistor, or so forth. Optical detector 8 may include optical filters, beam splitters, and so forth that may remove background light and reduce the total input optical signal at the detector 8 to one or more diagnostically relevant emission peaks. An input signal at detector 8 may be examined and analyzed for emission peaks of interest according to any suitable method. For instance, optical detector 8 may comprise a plurality of notch filters, each of which may be tuned to the spectral signature of a different autofluorescent pathogen. In one particular embodiment, the total input optical signal to detector 8 may be deconvoluted and analyzed according to a principal components analysis (PCA) regime as is known in the art.

For instance, input data to detector 8 may be reduced to relevant emission peaks based on maximum variations between the input spectra. In those embodiments in which a device is designed to examine a site for a plurality of different pathogens, the total input optical signal at the detector 8 may include a plurality of diagnostically relevant emission peaks. Accordingly, detector 8 may generate an output signal representing one or more emission peaks of interest. In addition, detector 8 may provide information with regard to the strength of each signal, for instance the pulses of light emitted over a particular time having a particular spectral signature, and this information may be correlated to the concentration of the detected pathogen.

In one particular embodiment, the signal from detector 8 may be transmitted to signal processor 12 for further analysis according to a PCA method. A PCA regime may utilize information regarding a library of spectra derived from pathogens, e.g., bacteria, of a reference sample to create a reference set, wherein each of the spectra are acquired under identical conditions. Data analysis techniques that may be carried out may include spectral data compression and linear regression. Using a linear combination of factors or principal components, a reconstructed spectrum may be derived. This reconstructed spectrum may then be compared with the spectra of known specimens which serve as the basis for determination of the presence or concentration of bacteria at the site of inquiry.

U.S. Pat. Nos. 7,110,886 to Ito, et al., 6,961,599 to Lambert, et al. and 6,662,621 to Cohenford, et al., all of which are incorporated herein by reference thereto, describe PCA regimes as may be utilized in analysis of an emission signal. In addition, a number of computer programs are available which carry out these statistical methods, including PCR-32™ (Bio-Rad, Cambridge, Mass., USA) and PLS-PLUS™ and DISCRIMINATE™ (Galactic Industries, Salem, N.H., USA). Discussions of the underlying theory and calculations of suitable methods may be found in, for example, Haaland, et al., Anal. Chem. 60:1193-1202 (1988); Cahn, et al., Applied Spectroscopy, 42:865-872 (1988); and Martens, et al., Multivariate Calibration, John Wiley and Sons, New York, N.Y. (1989).

Signal processor 12 may include a microprocessor configured to evaluate the strength or other characteristics of the output signal received over line 10 to, e.g., detect which specific bacteria is present in the field of enquiry and to produce a detection signal that may be coupled to line 14 for passage to a signaling device 16. Accordingly, if the detection signal reaches a predetermined threshold value, corresponding to a positive determination of the target pathogen, a detectable signal may be initiated at signaling device 16. For example, a detectable signal may be initiated at a signaling device 16 upon detection of any pathogen, i.e., any detection of a targeted pathogen at all may trigger initiation of a signal at signaling device 16. Optionally, if the detection signal at signal processor 12 indicates a pathogen concentration greater than a threshold amount, which may be correlated to the strength of the input signal to signal processor, signaling device 16 may be triggered to initiate a signal. For instance, signaling device 16 may be preset to initiate a detectable signal when the strength of the emitted signal correlates to a bacterial concentration greater than about 10⁵ CFU/mL (colony forming units/milliliter), in one embodiment.

In an exemplary configuration, a detectable signal may initiate a visible or audible signal that may be detected by the wearer within or at the surface of the enclosure 20 by way of signaling device 16. For instance, a visible signal may optionally include utilization of a liquid crystal diode (LCD) device, or an equivalent thereof, that may provide the signal as a readable output. For example, a visual signal may be provided at a surface of the device as an instruction such as, for instance, “CALL YOUR DOCTOR”, “VISIT HOSPITAL,” or so forth.

In addition to or alternative to a visual and/or audible signal at the enclosure 20 itself, signaling device 16 may include a transmitter portion that, upon initiation of the detectable signal, may transmit an electromagnetic signal to receiver 18. Receiver 18 may be remote from the signaling device 16. For instance, receiver 18 may be on the wearer's body at a distance from the signaling device 16, at a location apart from the wearer's body that may be conveniently chosen by the wearer, e.g., within the wearer's home, office, or so forth, or may be at a monitoring facility, for instance at a medical facility, such that appropriate medical personal may be quickly informed of the change in status of the patient's site of inquiry. In alternative embodiments, the detectable signal may be transmitted to multiple receivers, so as to inform both the wearer and others (e.g., medical personnel) of the change in status of a site. Transmission of a signal to a remote site may be carried out with a radio frequency transmission scheme or with any other wireless-type transmission scheme, as is generally known in the art. For instance, a wireless telephone or internet communications scheme could be utilized to transmit a signal to a remote location according to known methods.

Wireless transmission systems as may be utilized in conjunction with disclosed devices and methods may include, for example, components and systems as disclosed in U.S. Pat. Nos. 6,289,238 to Besson, et al., 6,441,747 to Khair, et al., 6,802,811 to Slepian, 6,659,947 to Carter, et al., and 7,294,105 to Islam, all of which are incorporated in their entirety by reference.

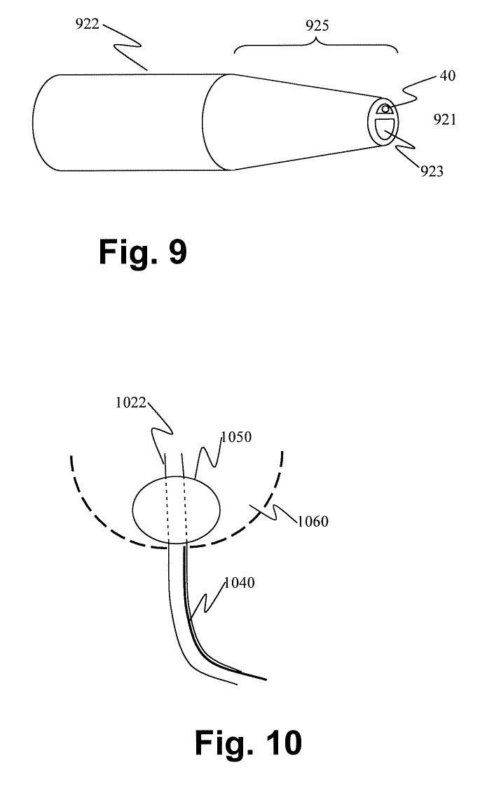

As previously mentioned, sensors as described herein are not limited to devices for use in drainage of a surgical or wound site. Another embodiment of a sensing device as disclosed herein is illustrated in FIG. 9. According to this embodiment, catheter 922 may be designed for use as an intravenous catheter. For instance, catheter 922 may be of a size and shape for use as a pulmonary artery catheter, a peripherally inserted catheter, a central venous catheter, or so forth. An intravenous catheter 922 may generally include a tapered distal end 925 for insertion into a blood vessel, for instance according to the Seldinger technique. For instance, distal end 925 of catheter 922 may be of a single construction or may be constructed separately from the catheter body.

An intravenous catheter 922 may be formed fairly flexible, so as to be easily inserted into and pass through the venous architecture without damaging the vessel walls. For example, a venous catheter 922 may be formed of soft, flexible polyurethane such as Tecoflex® or Pellethane®.

Intravenous catheter 922 is a multi-lumen catheter including lumen 923, through which a fluid may flow, for instance for delivery into an artery or vein, and also including lumen 921, within which fiber optic cable 40 may be affixed, for instance with an adhesive or through any other suitable bonding methodology. Following insertion, an optically detectable signal emitted by a pathogen may be transmitted by fiber optic cable 40 to a monitor, as described above. For instance, in one embodiment, fiber optic cable 40 may deliver an excitation signal to the site that may excite an optically detectable emission signal by targeted pathogenic bacteria in the field of inquiry. The autofluorescent emission signal may then be transmitted back to a monitor via fiber optic cable 40.

Of course, an intravenous catheter as described herein does not require a multi-lumen catheter, as is illustrated in FIG. 9. In other embodiments, an intravenous catheter may be a single lumen catheter or may include additional lumens, for instance for insertion of a temperature sensor, a pH sensor, and so forth through a lumen. Similarly, other types of catheters described herein may alternatively be multi-lumen or single lumen catheters, as desired.

In another embodiment, a sensor may include a fiber optic cable in conjunction for a Foley catheter or a ureteral catheter, for instance in conjunction with bladder and/or kidney drainage in detection of a hospital acquired urinary tract infection. FIG. 10 illustrates a sensor including a fiber optic cable 1040 in conjunction with a Foley catheter 1022. Foley catheter 1022 includes a balloon 1050, which is inflated following insertion of the catheter 1022 and used to hold the catheter in place in the bladder 1060. According to the illustrated embodiment, Foley catheter 1022 may incorporate a fiber optic cable 1040. For instance, one end of fiber optic cable 1040 may be located so as to detect and carry an optically detectable emission caused due to the presence of pathogenic bacteria in the area. In the example of the illustrated embodiment of FIG. 10, fiber optic cable 1040 is located so as to detect the presence of pathogen at the base of the bladder 1060, and below the base of balloon 1050. As such, any fluid that may collect at the base of the bladder 1060 may be examined for improved early detection of infection.

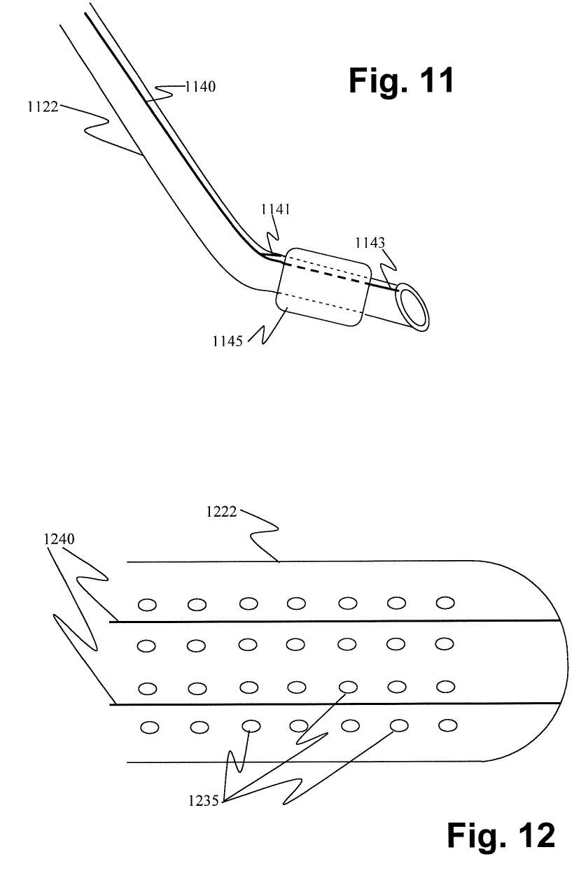

In yet another embodiment, a sensor may include a fiber optic cable in conjunction with an endotracheal tube 1122, as illustrated in FIG. 11. As is known, an intubated patient is placed at risk by the accumulation of pooled secretions between the inflated cuff 1145 and the oral pharyngeal area. The accumulation and stagnation of oral secretions breeds infectious organisms that may result in pneumonia. Specifically, the accumulated secretions may leak into the patient's lungs or find their way into the lungs when the endotracheal cuff is deflated for removal or when a patient is turned or coughs, leading to pneumonia.

Referring to FIG. 11, a fiber optic cable 1140 is associated with endotracheal tube 1122. Fiber optic cable 1140 includes a first end portion 1141, which may include a single or multiple optical fibers. End portion 1141 terminates above cuff 1145, and may be utilized to examine the local environment within the trachea and above cuff 1145 where secretions may pool leading to and provide an environment conducive for the development of pathogens. Fiber optic cable 1140 also includes a second end portion 1143 that extends to the terminus of endotracheal tube 1122 for examination of the local area for the presence of pathogens. Accordingly, multiple areas along the length of the device may be examined for the early detection of infection.

In yet another embodiment, a sensor may include a fiber optic cable in conjunction with a catheter utilized for delivery of pain medication, for instance directly to a surgery site. FIG. 12 illustrates one such embodiment of a pain release catheter including catheter 1222 and multiple fiber optic cables 1240. During operation, catheter 1222 may be inserted into a wound site. An infusion pump (not shown) may be loaded with a drug that may then be pumped through catheter 1222 and released through a series of apertures 1235 into the local environment. A plurality of fiber optic cables 1240 may be utilized to examine the local environment along the length of the catheter 1222 for optically detectable signals signifying the presence of pathogens that may cause infection.

While the subject matter has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present disclosure should be assessed as that of the appended claims and any equivalents thereto.

What is claimed is:

1. A method for detecting the presence or amount of a pathogen that is a source of a hospital acquired infection comprising:
locating a portion of an implantable device in an in vivo environment;
transmitting an optically detectable signal that is directly or indirectly emitted from the pathogen through a fiber optic cable to a detector, wherein the fiber optic cable is held in conjunction with the implantable device; and
determining the presence or amount of the pathogen in the environment.

2. The method according to claim 1, further comprising transmitting an excitation signal to the pathogen.

3. The method according to claim 1, wherein the optically detectable emission signal is an autofluorescent emission of the pathogen.

4. The method according to claim 1, wherein the implantable device comprises a catheter.

5. The method according to claim 4, further comprising transporting a fluid through the catheter from the in vivo environment to a reservoir, wherein the fluid includes the pathogen.

6. The method according to claim 5, wherein the optically detectable emission signal is emitted from the pathogen in the reservoir.

7. The method according to claim 5, further comprising concentrating the proportion of the pathogen in the fluid.

8. The method according to claim 4, further comprising transporting a fluid through the catheter to the in vivo environment.

9. The method according to claim 1, wherein the in vivo environment is a surgical site.

10. A device for detecting the presence or amount of a pathogen that is a source of a hospital acquired infection comprising:
an implantable device;
a fiber optic cable affixed to the implantable device, the fiber optic cable comprising a first end for location in an environment containing the pathogen and a second end in optical communication with an optical detector;
a portable enclosure containing a power source, the optical detector, a signal processor, and a signaling device for emitting a signal upon detection of the presence or amount of the pathogen in the environment; and
a connecting device for attaching the enclosure to the clothing or body of a wearer.

11. The device of claim 10, the enclosure further including a transmitter for transmitting a signal containing information regarding the presence or amount of the bacteria in the environment to a receiver.

12. The device of claim 11, wherein the transmitter is a wireless transmitter.

13. The device of claim 10, the enclosure further containing an excitation energy source for providing an excitation signal to the environment.

14. The device of claim 10, the fiber optic cable comprising a first optical fiber in optical communication with the excitation energy source, and a second optical fiber in optical communication with the optical detector.

15. The device of claim 10, wherein the connecting device is for connecting the enclosure to a piece of clothing.

16. The device of claim 10, wherein the connecting device is for connecting the enclosure to a wearer's skin.

17. The device of claim 10, wherein the implantable medical device comprises a catheter.

18. The device of claim 17, wherein the catheter is a pain release catheter.

19. The device of claim 18, wherein the catheter is a drainage tube.

20. The device of claim 18, wherein the catheter is a venous catheter.

21. The device of claim 10, wherein the implantable medical device is an endotracheal tube.

22. The device of claim 10, wherein a portion of the fiber optic cable is held within a wall of the implantable device.

23. The device of claim 10, wherein the implantable device is a multi-lumen device.

24. The device of claim 23, wherein a portion of the fiber optic cable is held within a lumen of the implantable device.
